(19) United States
(12) Patent Application Publication
(10) Pub. No.: US 2003/0017503 A1
(43) Pub. Date: Jan. 23, 2003

(54) Methods and labeled molecules for determining ligand binding to steroid receptors

(75) Inventors: Susan E. Goldrick, Danbury, CT (US); Richard M. Nelson, Newtown, CT (US); James J. Crute, San Diego, CA (US); Ruby C. Wasti, Bethel, CT (US); Gerald H. Nabozny, New Milford, CT (US); John R. Proudfoot, Newtown, CT (US); David S. Thomson, Ridgefield, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT (US)

(21) Appl. No.: 10/151,133

(22) Filed: May 20, 2002

Related U.S. Application Data
(60) Provisional application No. 60/291,877, filed on May 18, 2001.

(51) Int. Cl. G01N 33/53; C07J 17/00

(52) U.S. Cl. 435/7.1; 540/114

(57) ABSTRACT

The invention relates to fluorescence polarization (FP) methods for detecting and evaluating ligand binding to steroid receptors which are associated with heat shock proteins (hsps). The invention also relates to novel labeled molecules, in particular, fluorescence probes, which are useful in the methods of the invention.

### RELATED APPLICATIONS

This application claims priority benefit to U.S. Provisional Application serial No. 60/291,877 filed May 18, 2001.

### BACKGROUND OF THE INVENTION

The invention relates to fluorescence polarization (FP) methods for detecting and evaluating ligand binding to steroid receptors which are associated with heat shock proteins (hsps). The invention also relates to novel fluorescence probes which are useful in the methods of the invention.

Steroids and related hormones play an important role in regulating development, differentiation and homeostasis. There are five major classes of steroid hormones: progestins, glucocorticoids, mineralocorticoids, androgens and estrogens. The hormones exert their regulatory effects by binding to a superfamily of intracellular receptors, which are direct modulators of gene transcription.

Endogenous glucocorticoids play an important role in the normal regulation of the immune system and act as physiological immunosuppressants involved in the control of immune and inflammatory hyperactivity during the stress response (Munck et al., Physiological functions of glucocorticoids in stress and their relation to pharmacological actions, Endocr. Rev., 5:25-44, 1984). Glucocorticoids, at pharmacological dosages, are one of the principal therapeutics in the treatment of a large number of inflammatory and immunologically mediated disorders, including allograft rejection and autoimmune diseases (Axelrod, Glucocorticoid Therapy, Medicine (Baltimore) 55:39-65, 1976).

Unactivated steroid receptors are found in the cytosol where they are complexed with other proteins including heat shock proteins (Kimmins and MacRae, Maturation of steroid receptors: an example of functional cooperation among molecular chaperones and their associated proteins, Cell Stress Chaperones, 5(2):76-86, 2000; Lebeau et al., P59, an hsp90-binding protein, J. Biol. Chem. 267:4281-4284, 1992). Studies in which the chaperone machinery is assembled on the receptor in a stepwise fashion indicate that activation of steroid binding to the glucocorticoid receptor (GR) requires the co-presence of heat shock proteins (hsps) hsp90 and hsp70 while hsp organizer protein (Hop), hsp40, and p23 act as co-chaperones to enhance activation and assembly (Morishima , Y. et al., J. Biol. Chem. 275:18054-18060, 2000; Dittmaretal., J. Biol. Chem. 272(34):21213-21220, 1997). After binding of the steroid, the accessory proteins dissociate and the occupied receptor translocates into the nucleus. Once inside the nucleus, the receptor-steroid complex binds to specific sequences in the 5′ flanking regions of target genes and alters the transcriptional activity of these genes. Interaction with these sequences can inhibit as well as promote gene transcription (Muller and Renkawitz, The glucocorticoid receptor, Biochim. Biophys. Acta 1088:171-182, 1991; Gronemeyer, Control of transcription activation by steroid hormone receptors, FASEB J. 6:2524-2529, 1992).

Alnemri and Litwack examined the co-expression of hsp70 and hsp90 with GR and mineralocorticoid receptor (MR) in the baculovirus expression system (Alnermi and Litwack, Biochem. 32:5387-5393, 1993). However, their attempts to assemble the GR and MR in vivo by co-expression of the receptors with hsp90 or hsp70 failed to cause any increase in the formation of the steroid binding activity over GR or MR alone (Alnemri and Litwack, Biochem. 32:5387-5393, 1993).

Traditional standard assays commonly utilize radiolabeled ligands; these assays are cumbersome and labor intensive. Furthermore, traditional assays are only stable at 4° C.

Accordingly, there is a great need in the art for sensitive, stable methods to reliably detect ligand binding to steroid receptors. In addition, there is a need for a non-radioactive probe for use in such methods.

The fluorescence polarization assay of the present invention is a very sensitive and highly reproducible assay. This facilitates the determination of structure-activity relationships and the ranking of closely related test ligands. It also has a very high signal to noise ratio, is not subject to auto-hydrolysis since it is not an enzyme assay, and is amenable to high throughput screening.

Citation of identification of any reference in this section or any other part of this specification shall not be construed as an admission that such reference is available as prior art to the present invention.

### SUMMARY OF THE INVENTION

The present invention is directed to an FP assay for detecting and evaluating ligand binding to steroid receptors using labeled molecules that specifically bind the steroid receptor of interest. The present invention is based, in part, on Applicants' unexpected discovery that a steroid receptor co-expressed with hsps produces a sensitive, reproducible assay that is stable at room temperature for at least 24 hours.

Another key feature of the FP assay of the present invention is the use of novel fluorescence probes that bind to the steroid receptor of interest.

The fluorescence polarization assay of the present invention generally comprises the following steps:

(a) determining the fluorescence polarization values of the free fluorescent probe and the fluorescent probe bound to an expression vector lysate, wherein the lysate comprises a steroid receptor associated with at least three heat shock proteins (hsps) to obtain a range of fluorescence polarization values and selecting a reference fluorescence polarization value falling within that range;

(b) mixing the fluorescent probe with the lysate in step (a) in a buffered aqueous solution;

(c) mixing a test compound with the mixture obtained in step (b) and incubating the resulting mixture of fluorescent probe, lysate, and test compound;

(d) measuring the fluorescence polarization value of the incubated mixture obtained in step (c) to obtain a test fluorescence polarization value; and

(e) determining the difference between the test fluorescence polarization value and the reference fluorescence polarization value;

wherein the difference in fluorescence polarization values obtained in step (e) indicates whether the test compound binds the steroid receptor.

The assay of the present invention is very sensitive and highly reproducible and can detect compounds that positively or negatively affect probe binding to the steroid receptor by analyzing corresponding changes in fluorescence polarization. This assay can also be used in high throughput screening procedures, e.g., efficiently screening a library of test compounds for steroid receptor binding activity.

In a preferred embodiment, the expression vector is a baculovirus system.

Preferably, the steroid receptor is GR, MR, androgen receptor (AR) or estrogen receptor (ER). More preferably, the steroid receptor is GR or MR. Preferably, the hsps are at least hsp90, hsp70 and p23.

In an embodiment, the labeled molecule is a fluorescently-labeled probe (also named herein, “fluorescence probe”) wherein the fluorescent label is rhodamine or a rhodamine derivative.

In a preferred embodiment, the labeled molecule is labeled with tetramethyl rhodamine (TAMRA). In a preferred embodiment, the molecule is mifepristone (RU-486) or a derivitive thereof. In another embodiment, the molecule is dexamethasone.

### BRIEF DESCRIPTION OF THE DRAWINGS

FIG. 1 shows the fluorescence polarization results obtained by titrating free probe (5 nM, TAMRA-RU-486) with a hypotonic lysate containing the glucocorticoid receptor (GR) (▪), or a hypotonic lysate which does not contain the GR (●), or a hypotonic lysate containing the GR in the presence of 500 nM dexamethasone (▴).

FIG. 2 shows binding of GR-ligand binding domain (LBD) tagged with glutathione transferase (GST) to ³H-dexamethasone in the presence of p23 (grey with black dots); p23 in combination with hsp 90 (black with white dots); and p23 in combination with hsp 70 and hsp90 (diagonal stripes). Control was GST/GR-LBD in the absence of hsp (vertical stripes).

FIG. 3 shows the LBD of GR tagged with GST (SEQ ID NO: 1). Compared to full-length GR, this construct contains amino acids 1-226 and 747-1003. The GST tag adds 2 additional amino acids, alanine and methionine (underlined). The * indicates the presence of a GST tag.

FIG. 4 shows full-length GR (SEQ ID NO: 2). The * indicates the presence of a GST tag.

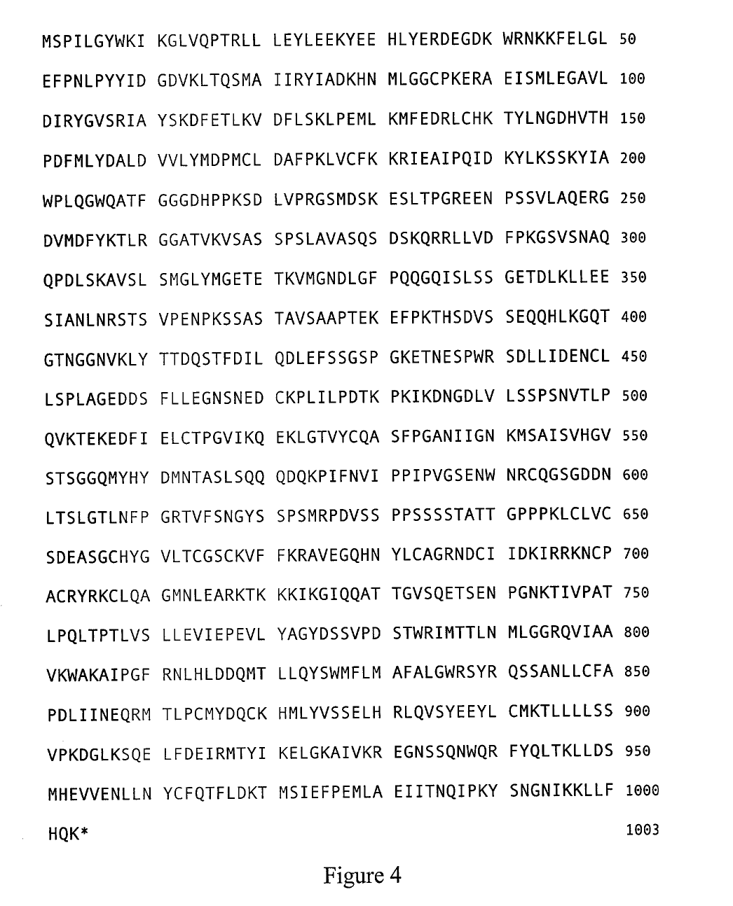

### DETAILED DESCRIPTION OF THE INVENTION

I. The Fluorescent Probes

The fluorescent probes of the present invention comprise molecules which bind to steroid receptors. Such molecules are well known in the art and include, but are not limited to dexamethasone, mifepristone (RU-486) and derivatives thereof. Modifications of mifepristone may include, but are not limited to modifications of C 17 on the D ring and modification of C3 on the A ring. Several molecules and derivatives are commercially available.

In an embodiment, dexamethasone is fluorescently labeled. In another embodiment, RU-486 or derivatives thereof are fluorescently labeled.

Fluorescent labels suitable for use in the invention include any of those well known in the art. See, for example, those described in “Handbook of Fluorescent Probes and Research Chemicals” by Richard P. Haugland, Sixth edition (1996). The eighth edition is available on CD-ROM and an updated seventh edition is available on the Web at www.probes.com/handbook/. A number of suitable fluorescent labels are commercially available from Molecular Probes, Inc. (Eugene, Oreg.) It is preferred that the fluorescent label fluoresces at a relatively high wavelength, ie., above about 450 nm; to avoid interference from cell originating fluorescence and fluorescence originating from test compounds and impurities present in the system or from glass and plastic containers. Accordingly, in one embodiment, the fluorescent label of the invention fluoresces at a wavelength above about 450 nm. More preferably, the label fluoresces above about 550 nm and less than about 700 nm.

Examples of fluorescent labels useful in the present invention include rhodamine and rhodamine derivatives such as tetramethyl rhodamine, carboxytetramethylrhodamine, Lissamine™ Rhodamine B, Texas Red®, carboxy-X-rhodamine and Rhodamine Red™-X, and other rhodamine derivatives known in the art, fluorescein and fluorescein derivatives such as fluorinated fluoresceins such as Oregon Green® and its derivatives, fluoresceinamine, carboxyfluorescein, alpha-iodoacetamidofluorescein, 4′-aminomethylfluorescein, 4′-N-alkylaminomethylfluorescein, 5-aminomethylfluorescein, 6-aminomethylfluorescein, 2,4-dichloro-1,3,5-triazin-2-yl-aminofluorescein (DTAF), 4-chloro-6-methoxy-1,3,5-triazln-2-yl-aminofluorescein, and fluoresceinisothiocyanate, and other fluorescein derivatives known in the art, 4,4-difluor-4-bora-3a,4a-diaza-s-indacene and its derivatives, cyanine dyes, and the Alexa Fluor® dyes.

In a preferred embodiment, the fluorescent label is tetramethyl rhodamine (TAMRA).

Fluorescent probes of the invention may be prepared by methods well known in the art. Optimum reaction conditions and reaction times may vary depending on the particular reactants used. Unless otherwise specified, solvents, temperatures, pressures and other reaction conditions may be readily selected by one of ordinary skill in the art. A specific procedure, for illustrative purposes, is provided in the Examples section. Typically, reaction progress may be monitored by thin layer chromatography (TLC) if desired. Intermediates and products may be purified by chromatography on silica gel and/or recrystallization. Starting materials and reagents are either commercially available or may be prepared by one skilled in the art using methods described in the chemical literature.

II. The Fluorescence Polarization Assay

Fluorescence polarization immunoassay procedures have been used to provide a reliable quantitative means for measuring the amount of probe-receptor complex produced in a competitive binding assay. Typically, in such a competitive binding assay a ligand (a substance of biological interest to be determined by the technique) competes with a fluorescently labeled reagent, or “ligand analog” or “probe”, for a limited number of receptors specific to the ligand and ligand analog. The concentration of ligand in the sample determines the amount of ligand analog which binds to the receptor: the amount of ligand analog that will bind is inversely proportional to the concentration of ligand in the sample, because the ligand and the ligand analog each bind to the receptor in proportion to their respective concentrations.

Fluorescence polarization techniques are based on the principle that a fluorescently labeled compound, when excited by plane polarized light, will emit fluorescence having a degree of polarization inversely related to its rate of rotation. Accordingly, when a probe-receptor complex having a fluorescent label, for example, is excited with plane polarized light, the emitted light remains highly polarized because the fluorophore is constrained from rotating between the time that light is absorbed and emitted. In contrast, when a “free” probe compound (i.e., unbound to a receptor) is excited by plane polarized light, its rotation is much faster than that of the corresponding probe-receptor conjugate and the molecules are more randomly oriented. As a result, the light emitted from the unbound probe molecules is depolarized.

The present inventors have discovered that the novel fluorescent probes of the present invention can be used in a fluorescence polarization assay to detect and evaluate ligands which bind to steroid receptors. The fluorescent probes of the present invention specifically bind to the steroid receptor of interest. Upon complexing with the steroid receptor, the probe-receptor complex thus formed assumes the rotation of the receptor molecule which is slower than that of the relatively small fluorescent probe molecule, thereby increasing the polarization observed. When a test compound competes with the fluorescent probe for binding to the receptor, less probe-receptor complex is formed, i.e., there is more probe in an uncomplexed, free form. Therefore, the observed polarization of fluorescence of the resulting mixture of free probe and probe-receptor complex assumes a value intermediate between that of the free probe and that of the probe-receptor complex. Thus, there is a reduction of the fluorescence polarization value in the presence of a competitive inhibitor of receptor ligand as compared to when no such inhibitor is present. Inhibitor dissociation constants can then be easily determined in order to evaluate the relative strength of the competitive inhibitor.

The fluorescent probes of the invention can also be used to detect and evaluate non-competitive inhibitors of steroid receptors, e.g., allosteric inhibitors, that bind to a site on the steroid receptor molecule other than the active site but affect binding at the active site. The effect of the non-competitive inhibitor on active site binding, either positive or negative, can be detected in the assay by corresponding changes in the fluorescence polarization value, said changes demonstrating either enhancement or suppression of probe binding at the active site.

The fluorescent probes of the invention can also be used to determine protein expression levels of steroid receptors. In a preferred embodiment, the fluorescently-labeled probes are used to determine GR or MR expression levels.

The steroid receptor used in the methods and probes of the invention may contain a tag, including but not limited to, glutathione transferase (GST).

Unless otherwise specified herein, the conditions that can be employed in running the fluorescence polarization assays of the present invention (e.g., pressure, temperature, pH, solvents, time) may be readily determined by one having ordinary skill in the art. Of course, the optimum assay conditions may vary depending on the particular reagents used (i.e., the fluorescent probe, the expression vector lysate, and the test compound) and such optimum conditions can also be readily determined by one skilled in the art based on the general knowledge in the field of fluorescence polarization.

In one embodiment, the fluorescence polarization assay of the present invention comprises the following steps:

(a) determining the fluorescence polarization values of a free fluorescently-labeled probe and the fluorescently-labeled probe bound to an expression vector lysate wherein the lysate comprises a steroid receptor associated with at least three heat shock proteins (hsps) to obtain a range of fluorescence polarization values and selecting a reference fluorescence polarization value falling within that range;

(b) mixing the fluorescently-labeled probe with the lysate in step (a) in a buffered aqueous solution;

(c) mixing a test compound with the mixture obtained in step (b) and incubating the resulting mixture of fluorescently-labeled probe, lysate, and test compound;

(d) measuring the fluorescence polarization value of the incubated mixture obtained in step (c) to obtain a test fluorescence polarization value; and

(e) determining the difference between the test fluorescence polarization value and the reference fluorescence polarization value;

wherein the difference in fluorescence polarization values obtained in step (e) indicates whether the test compound binds the steroid receptor.

As a preliminary step, it is desirable to determine the wavelengths of maximum excitation and emission of the particular fluorescent probe selected to be used in the assay, unless these values are already known. These wavelengths can be determined using any conventional technique, for example, by measuring the respective excitation and emission wavelengths of the probe in a suitable assay buffer using a fluorometer.

In step (a) of the assay, the affinity of the fluorescent probe for the steroid receptor is determined by measuring the fluorescence polarization values of the free (unbound) fluorescent probe and the fluorescent probe bound to the steroid receptor to obtain a range of fluorescence polarization values. The polarization value of the free fluorescent probe would usually be the minimum value in this range and, likewise, the polarization value of the bound fluorescent probe would usually be the maximum value in this range. In one embodiment, this range of fluorescence polarization values in step (a) is obtained by periodically adding increasing amounts of expression vector lysate to an amount of fluorescent probe in a buffered aqueous solution, for example, by titration, and then measuring the fluorescence polarization value of this mixture after each addition of expression vector lysate until no further significant change in polarization value is observed. If desired, one may then use the data obtained in conjunction with conventional methods (e.g., regression analysis) to calculate the dissociation constant of the fluorescent probe for the steroid receptor.

From the results obtained in step (a), one can then select an appropriate reference fluorescence polarization value for use in the assay, this reference fluorescence polarization value falling in the range of polarization values obtained in step (a). One skilled in the art can best determine the particular reference polarization value to use in the assay, depending on the affinity of the specific fluorescent probe for the steroid receptor, the expected inhibitory strength of the test compound, and other conditions and variables.

In general, however, the reference fluorescence polarization value is selected such that it falls within the upper half of the range of polarization values obtained in step (a). For example, the reference fluorescence polarization value may be selected such that the difference between the reference fluorescence polarization value and the polarization value of free fluorescent probe is equal to about 50% to 100%, preferably about 80% to 100%, of the difference between the polarization value of fluorescent probe bound to the steroid receptor and the polarization value of free fluorescent probe.

In the next step (b), the fluorescent probe is mixed with the lysate in a buffered aqueous solution in order to form a complex between the fluorescent probe and the steroid receptor. The concentrations of the fluorescent probe and the lysate should be chosen so as to facilitate competition between the probe and the test compounds for binding to the steroid receptor and will depend on a number of factors including the binding affinity of the probe for the steroid receptor. The appropriate concentrations to use in a particular assay can be readily determined by one skilled in the art.

In the next step (c), a test compound is mixed with the fluorescent probe-lysate complex mixture obtained in step (b), and the resulting mixture of fluorescent probe lysate and test compound is incubated to facilitate competition or other interaction. In one embodiment, the test compound may be dissolved in a buffered aqueous solution prior to mixing it with the probe-lysate mixture. If the test compound is water-insoluble, it may be necessary to first dissolve the test compound in an appropriate organic solvent, for example, DMSO (dimethyl sulfoxide), prior to diluting it in the buffered aqueous solution. If an organic solvent is used, the final percent organic solvent in the assay mixture should not exceed about 1%. The incubation conditions for this step can vary, but generally the incubation is conducted at a temperature of about 25° C. for about 15 minutes.

The fluorescence polarization value of the incubated mixture is then measured, step (d), in order to obtain a test fluorescence polarization value. The fluorescence polarization can be measured using well-known techniques in the art, as described hereinafter. For example, the polarization can be measured using a fluorescence polarization plate reader set at the wavelength appropriate for the fluorescent label on the fluorescent probe. The difference between the test fluorescence polarization value obtained in step (d) and the reference fluorescence polarization value will then indicate whether the test compound binds steroid receptor and the relative strength of the binding effect, if any.

When the difference in fluorescence polarization values obtained in step (d) is positive, i.e., there is an increase in the polarization in the presence of test compound, this could indicate that the test compound is a non-competitive (allosteric) inhibitor. Where the difference in fluorescence polarization values obtained in step (d) is negative, i.e., there is a decrease in the polarization in the presence of test compound, this could indicate that the test compound is a competitive inhibitor that competes with the fluorescent probe for active site binding on the steroid receptor.

When the assay is run using multiple dilutions of a test compound, the range of test fluorescence polarization values obtained can be plotted on an appropriate graph. If desired, one may then use conventional methods (e.g., regression analysis) to calculate the dissociation constant of the test compound for binding to the steroid receptor.

The assay of the present invention can be run at a wide range of pH levels. In general, the pH may range from about 3 to 12, more usually from about 5 to 10, preferably from about 5 to 8. Various buffers may be used to achieve and maintain the pH during the assay procedure. Representative buffers for use in the assay include borate, phosphate, carbonate, TRIS (2-[(2-hydroxy-1,1-bis[hydroxymethyl]ethyl)amino]ethanesulfonic acid), TES (2-amino-2-hydroxymethyl-1,3-propanediol), and the like. The salt concentration of the buffer may fall within a wide range, but preferably the salt concentration is between 0 and about 600 mM. The buffered aqueous solution preferably further contains a reducing agent such as dithiothreitol (DTT) and it is preferred that the buffer contains a detergent, such as CHAPS (3[3-cholamidopropyl)-dimethylammonio]-1-propanesulfonate) or any other conventional detergent normally used in buffers. Within these parameters the particular buffer employed is not critical to the present invention, but in an individual assay a specific buffer may be preferred in view of the other conditions and reagents employed, as can readily be determined by one skilled in the art.

As discussed above, the fluorescence polarization values can be measured using techniques that are well known in the art. For example, by measuring the vertically and horizontally polarized components of the emitted light, the polarization of fluorescence in the reaction mixture may be accurately determined. (See Chapter 10 in “Principals of Fluorescence Spectroscopy” Second edition, J. R. Lakowizc, Kluwer Academic/Plenum Publishers, New York 1999 for detailed description of measurement).

The assay can be run using lysate of expression vectors which express steroid receptor from a variety of species. Preferably, the steroid receptor is a mammalian steroid receptor, for example, human or murine steroid receptor.

In another embodiment, the fluorescence polarization assay of the present invention can be employed to quickly and efficiently screen a library of test compounds for steroid receptor binding. This assay comprises the following steps:

(a) determining the fluorescence polarization values of a free fluorescently-labeled probe and the fluorescently-labeled probe bound to an expression vector lysate wherein the lysate comprises a steroid receptor associated with at least three heat shock proteins (hsps) to obtain a range of fluorescence polarization values and selecting a reference fluorescence polarization value falling within that range;

(b) mixing the fluorescently-labeled probe with the lysate in step (a) in a buffered aqueous solution;

(c) adding test compounds to a plurality of containers;

(d) adding the mixture obtained in step (b) to said plurality of containers, and incubating the resulting mixtures of fluorescently-labeled probe, lysate, and test compounds;

(e) measuring the fluorescence polarization values of the incubated mixtures obtained in step (d) to obtain test fluorescence polarization values; and

(f) determining the differences between the test fluorescence polarization values and the reference fluorescence polarization value;

wherein the differences in fluorescence polarization values obtained in step (f) indicate whether the test compounds bind steroid receptor.

Any of the conventional techniques and equipment known in the art for screening a large number of compounds (e.g., automated library screening) can be employed in this screening assay of the present invention. The plurality of containers used to hold the test compounds can take a variety of forms, for example, any of the conventionally used well plates for automated library screening. In one embodiment of the assay, the test compounds are diluted in a buffered aqueous solution prior to adding them to the plurality of containers. The general conditions, techniques, etc., employed in conducting this library screening assay are otherwise the same as discussed in detail above for the general assay method.

III. Expression of Steroid Receptor Associated with hsps

Methods which are well known to those skilled in the art can be used to construct expression vectors containing coding sequences of a steroid receptor and hsps and appropriate transcriptional/translational control signals. These methods include in vitro recombination/genetic recombination. See, for example, the techniques described in Sambrook et al., 1989, Molecular Cloning, A Laboratory Manual 2d ed., Cold Spring Harbor Laboratory, N.Y.

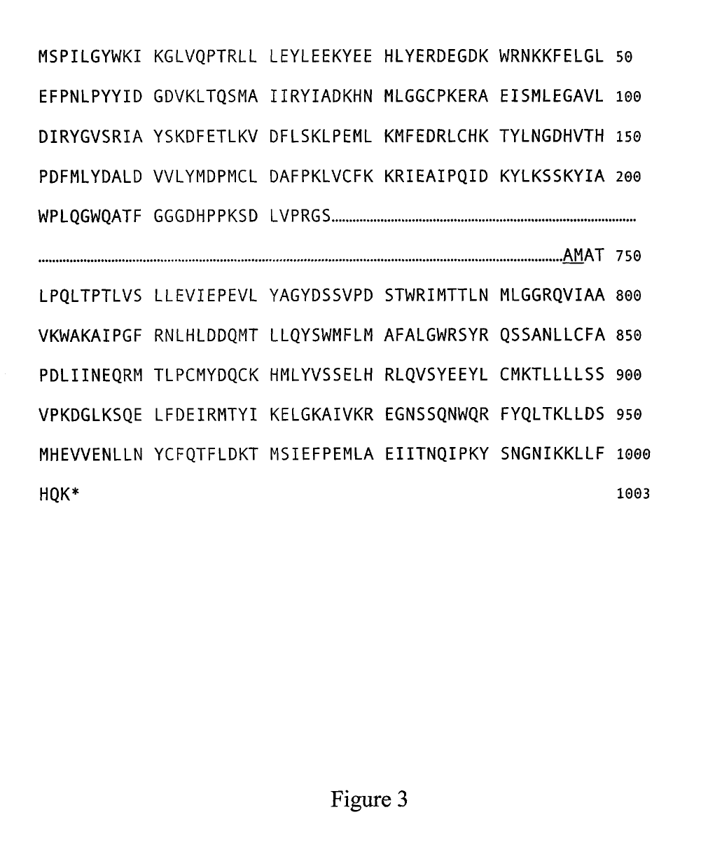

The invention also encompasses co-expression of one or more hsps and a modified steroid receptor. Preferably, the modified steroid receptor comprises the LBD. More preferably, the modified steroid receptor is the LBD of GR, see, for example, FIG. 3, SEQ ID NO: 1, or MR-LBD. The modified steroid receptor may contain a GST tag.

The modified steroid receptor may be made by methods well known in the art including recombinant techniques. See, for example, the techniques described in Sambrook et al., 1989, Molecular Cloning, A Laboratory Manual 2d ed., Cold Spring Harbor Laboratory, N.Y.

A variety of host-expression vector systems may be utilized to express coding sequences of a steroid receptor (or its LBD) and hsps including, but not limited to insect cell systems infected with recombinant virus expression vectors (e.g., baculovirus). Methods to express GR protein associated with hsps other than the baculovirus system include, but are not limited to, yeast (e.g. Picchia) or a mammalian cell line such as COS, HeLa or a variety of others. In both the yeast and mammalian systems, the cells can be lysed and the lysate used in a binding assay as described below using the baculovirus system.

In an embodiment, a steroid receptor is co-expressed with one or more hsps. Preferably, GR or MR is co-expressed with p23 and/or hsp90 and/or hsp70.

In addition to hsp90, hsp70 and p23, co-expression of additional hsps (e.g. hsp60) may also be employed to enhance hormone receptor binding.

Alternatively, hormone receptor binding may be re-constituted by co-incubating hormone receptor with hsp90, hsp70 and p23 derived from recombinant or cellular sources such as rabbit reticulocyte lysates (Dittmart et al. J. Biol. Chem. 272:21213-21220, 1997).

The following examples illustrate certain features of the present invention but are not intended to limit the scope of the present invention.

### EXAMPLES

Preparation of Recombinant Baculovirus for the Study of Steroid Receptors:

All of the necessary recombinant baculovirus, glucocorticoid receptor and the chaperone proteins, were prepared in the same manner. The DNA for each of the constructs was subcloned into a standard baculovirus transfer vector pVL1393 (BD PharMingen, San Diego, Calif.). Each DNA sample was then completely sequenced to verify the correct gene product.

Baculovirus preparation requires transfection and propagation of the gene of interest into insect cells. Each DNA sample was individually transfected into SF9 cells. Transfection via Lipofectin Reagent (Gibco/BRL, Invitrogen Life Technologies, Carlsbad, Calif.) occurs by homologous recombination between the gene of interest and Baculogold DNA (BD PharMingen, San Diego, Calif.). Samples are incubated for 5 days, and infectious virus is harvested. The transfection sample is then plaque purified and individual plaques are isolated and eluted.

Propagation of the recombinant virus is accomplished by amplification of several plaques to screen for protein production. This amplification is done twice, increasing both the cell number and the virus titer. Infected cells are lysed and nuclear and cytosolic fractions are prepared. Fractions are then analyzed by polyacrylamide gel electrophoresis and immunoblot to determine the level of expressed recombinant protein. A positive sample is selected, an additional amplification is performed, and a high titer stock is generated. Baculovirus stocks are then used alone and in combination to produce the necessary recombinant proteins.

Infection and Lysis of Infected Insect Cells for Recombinant Protein

Recombinant baculovirus stocks are used to infect insect cells for the production of the protein of interest. Small size infections are performed to determine the parameters for optimal expression. These specific parameters are then applied to prepared the larger amounts of recombinant protein necessary for research purposes.

Insect cells are infected at a density of between 7-9×10⁵ cells per ml. The overall cell number is critical for optimum infection. The cells are incubated at 27° C. shaking at 140 RPM for the desired amount of time. After the infection, cells are harvested by centrifugation at 3000 rpm for 15 minutes. The cell pellets are then rinsed with a protein free media and recentrifuged. The wash media is then gently removed leaving the cell pellet.

An estimate of the cell pellet size is made, and the cells are resuspended in 7 volumes of lysis buffer. The lysis buffer used for GR consisted of 20 mM HEPES (N-2-hydroxyethylpiperazine-N′-2-ethanesulfonic acid) pH 7.5, 1 mM DTT (dithiothreitol), 1 mM PMSF (phenylmethylsulfonyl fluoride), 10 mM sodium bisulfite, 10 ug/ml leupeptin, 10 ug/ml pepstatin, 4 mM magnesium chloride, 10 mM sodium molybdate and 1 mM ATP (Adenosine 5′-triphosphate). The resuspended cells are allowed to sit on ice for 10 minutes, then Dounce homogenized with a tight pestle 25 strokes to break the cells. A low speed centrifugation is performed to remove some of the cellular debris. The resulting supernatant is then centrifuged at 44,000 RPM for 75 minutes at 4° C. The supernatant fraction, or cytosolic fraction, is then divided into tubes for storage and quick frozen in liquid nitrogen. Fractions are then stored long term at −80° C.

Method for Fluorescent Labeling of Mifepristone:

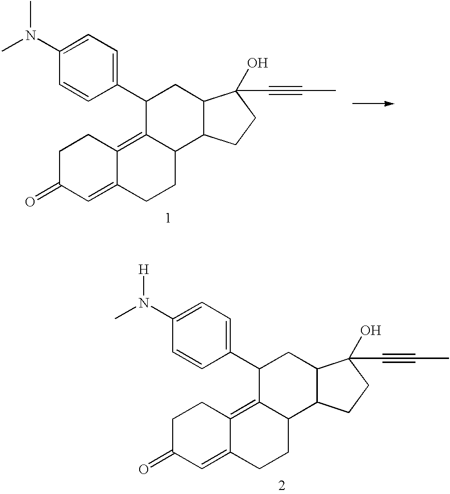

A mixture of mifepristone (Sigma, St. Louis, Mo.) 1 (RU-486) (0.10 g) and iodobenzene diacetate (0.08 g) in methylene chloride (0.5 mL) and acetonitrile (1 mL) was stirred at room temperature overnight. The product 2 (0.01 g) was obtained by purification over a silica gel column (eluent methylene chloride—methylene chloride/ethyl acetate gradient) followed by preparative layer chromatography (developer methylene chloride/ethyl acetate 10 /1).

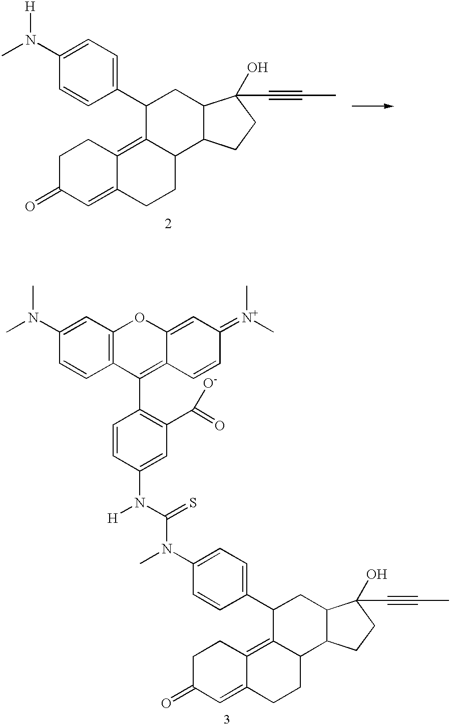

A mixture of 2 (0.001 g) and tetramethylrhodamine-5-isothiocyanate (5-TRITC, Molecular Probes, Inc., Eugene, Oreg., 0.002 g) in DMF (0. 1 mL was stirred at room temperature overnight. Additional 2 (0.005 g) and 5-TRITC (0.003 g) in methylene chloride (0.4 mL) and DMF (0.2 mL) was added and the mixture was left at room temperature for an additional 24 hours. The reaction mixture was fractionated directly over silica gel (eluent methylene chloride to acetone to acetone/ethanol gradient) to give 3 (0.0015 g).

Fluorescence Polarization Assay to Determine Binding of Steroid Receptor

Step One: Characterization of the Fluorescent Probe

The wavelengths for maximum excitation and emission of the fluorescent probe should first be measured. An example of such a probe is TAMRA-RU-486.

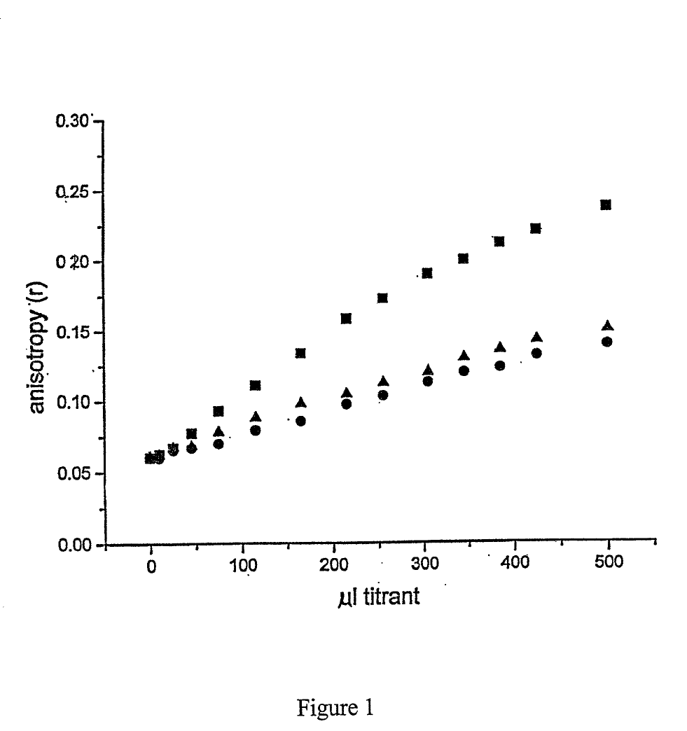

The affinity of the probe for the steroid receptor was then determined in a titration experiment. The fluorescence polarization value of the probe in assay buffer was measured on an SLM-8100 fluorometer using the excitation and emission maximum values described above. Aliquots of expression vector lysate were added and fluorescence polarization was measured after each addition until no further change in polarization value was observed. Non-linear least squares regression analysis was used to calculate the dissociation constant of the probe from the polarization values obtained for lysate binding to the probe. FIG. 1 shows the fluorescence polarization results obtained by titrating free probe (5 nM, TAMRA-RU-486) with a hypotonic lysate containing the glucocorticoid receptor (GR) (▪), or a hypotonic lysate which does not contain the GR (●), or a hypotonic lysate containing the GR in the presence of 500 nM dexamethasone (▴).

Step Two: Screening for Inhibitors of Probe Binding Using GR Competitive Binding Assay

This assay used fluorescence polarization (FP) to quantitate the ability of test compounds to compete with tetramethyl rhodamine (TAMRA)-labeled RU-486 for binding to a human glucocorticoid receptor (GR) complex. The receptor preparation was made from insect cells expressing human GR, hsp70, hsp90, and p23 as described above. The assay buffer was: 10 mM TES (2-amino-2-hydroxymethyl-1,3-propanediol), 50 mM KCl, 20 mM Na₂MoO₄.2H₂O, 1.5 mM EDTA (ethylenediaminetetraacetic acid), 0.04% w/v CHAPS, 10% v/v glycerol, 1 mM DTT, pH 7.4. Test compounds were dissolved to 1 mM in neat DMSO and then further diluted to 10×assay concentration in assay buffer supplemented with 10% v/v DMSO. Test compounds were serially diluted at 10×assay concentrations in 10% DMSO-containing buffer in 96-well polypropylene plates. Binding reaction mixtures were prepared in 96-well black Dynex microtiterrm plates (Dynex Technologies, Denkendorf, Germany) by sequential addition of the following assay components: 15 μL of each 10×test compound solution, 85 μL of GR-containing baculovirus lysate diluted 1:170 in assay buffer, and 50 μL of 15 nM TAMRA-labeled RU-486. Positive controls were reaction mixtures containing no test compound; negative controls (blanks) were reaction mixtures containing 1 μM dexamethasone. The binding reactions were incubated for 1 hour at room temperature and then read for FP in the LJL Analyst™ (LJL Biosystems, Molecular Devices Corp., Sunnyvale, Calif.) set to 550 nm excitation and 580 nm emission, with the Rh 561 dichroic mirror installed. IC₅₀ values were determined by iterative non-linear curve fitting of the FP signal data to a 4-parameter logistic equation.

Determination of Active GR Ligand by 3H-Dexamethasone

After expressing and preparing GR recombinant lysates containing various combinations of heat shock proteins, cytosolic fractions were analyzed for the ability to bind to ³H-dexamethasone.

The following assay was designed as a fast and efficient way to determine protein expression levels. Baculovirus cell lysates were diluted in a buffer consisting of the GR lysis buffer with 50 mM potassium chloride. Samples were diluted in buffer to yield 50 ul of final volume. ³H-dexamethasone was obtained from Perkin Elmer Life Sciences (Boston, Mass.). The specific activity range was between 35-50 Ci/mmol. A 1/10 dilution of dexamethasone was made in 2×Assay Buffer, which contains 40 mM Tris (pH 7.5), 20% glycerol, 5 mM sodium molybdate, 4 mM magnesium chloride, 2 mM ATP and 100 mM potassium chloride chilled. Fifty microliters of a 1/10 dilution of ³H-dexamethasone was added to 50 μl of cell lysate. The sample was mixed well and left at room temperature for 60 minutes.

To remove unbound 3H dexamethasone, 100 ul of 2% dextran coated charcoal (Sigma, St. Louis, Mo.) in 1×Assay buffer (described above) was added to each sample. The samples were mixed and left for 5 minutes. Each sample was centrifuged at 14,000 RPM for 2 minutes to remove the charcoal and unbound counts. One hundred sixty microliters of supernatant was removed from each sample to a fresh tube and 1 ml of Ready Safe Scintillation Cocktail (Beckman Coulter, Fullerton, Calif.) was added. Samples wer counted for bound ³H-dexamethasone on a Beckman LS5000TA scintillation counter. This protocol could be modified to incubate lysate and dexamethasone overnight at 4° C.

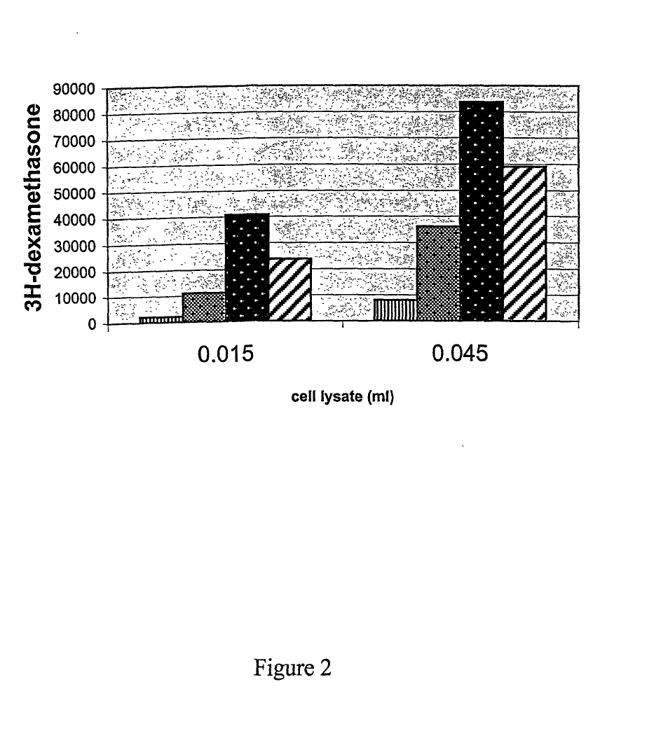

As shown in FIG. 2, the addition of various heat shock proteins to GST/GR-LBD dramatically increased expression levels compared to the control (GST/GR-LBD absent hsp).

GR FP UHTS Protocol

This ultra high throughput screen identifies compounds that inhibit the binding interaction of a human glucocorticoid receptor (GR) complex present in a baculovirus-infected insect cell lysate to a labeled probe, for example, tetramethyl rhodamine (TAMRA)-labeled RU-486 probe or TAMRA-labeled dexamethasone. The detection method is fluorescence polarization. The insect cells used to generate the receptor-containing lysates have been co-infected with 4 human proteins: GR, hsp70, hsp90, and p23. The UHTS employs the Zymark Allegro modular robotic system (Zymark Corp., Hopkinton, Mass.) to dispense reagents, buffers, and test compounds into either 96-well or 384-well black microtiter plates (from Dynex (Dynex Technologies, Denkendorf, Germany) or Corning (Corning Costar, Cambridge, Mass.), respectively). The assay buffer is: 10 mM TES, 50 mM KCl, 20 mM sodium molybdate, 1.5 mM EDTA, 0.04% w/v CHAPS, 10% v/v glycerol, 1 mM DTT, pH 7.4. For 384-well format, GR-containing baculovirus lysate is diluted 1 to 75 in cold assay buffer and 20 μL is added to each well. Test compounds dissolved in neat DMSO at 1 mg/mL are diluted to 80 μg/mL in assay buffer, and 10 μL of this dilution is added to the assay plate, for a final assay concentration of 10 μg/mL. TAMRA-labeled RU-486 or TAMRA-labeled dexamethasone is diluted to 8 nM in assay buffer, and 50 μL is added to the assay, for a final concentration of 5 nM and a final volume of 80 μL. Positive controls are reaction mixtures containing no test compound; negative controls (blanks) are reaction mixtures containing 1 μM dexamethasone. For 96-well format, the final concentration of all reaction components remains the same, the component volumes are doubled, and the final well volume is 160 μL. After incubating the reaction for 1 to 4 hours at room temperature, the plates are read for fluorescence polarization in the LJL Analyst™ set to 550 nm excitation, 580 nm emission, using the Rh 561 dichroic mirror.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings.

All publications cited herein are incorporated by reference in their entirety.

What is claimed is:

1. A fluorescence polarization assay for determining whether a compound binds steroid receptor comprising:
(a) determining the fluorescence polarization values of a free fluorescently-labeled probe and the fluorescently-labeled probe bound to an expression vector lysate, wherein the lysate comprises a steroid receptor associated with at least three heat shock proteins (hsps) to obtain a range of fluorescence polarization values and selecting a reference fluorescence polarization value falling within that range;
(b) mixing the fluorescently-labeled probe with the lysate in step (a) in a buffered aqueous solution;
(c) mixing a test compound with the mixture obtained in step (b) and incubating the resulting mixture of fluorescently-labeled probe, lysate, and test compound;
(d) measuring the fluorescence polarization value of the incubated mixture obtained in step (c) to obtain a test fluorescence polarization value; and
(e) determining the difference between the test fluorescence polarization value and the reference fluorescence polarization value;
wherein the difference in fluorescence polarization values obtained in step (e) indicates whether the test compound binds the steroid receptor.

2. The method according to claim 1, wherein the probe is dexamethasone or mifepristone.

3. The method according to claim 1 wherein the fluorescent label is rhodamine or a rhodamine derivative.

4. The method according to claim 1, wherein the fluorescently-labeled probe is tetramethylrhodamine-dexamethasone.

5. The method according to claim 1, wherein the fluorescently-labeled probe is tetramethylrhodamine-mifepristone.

6. The method according to claim 1, wherein the expression vector is selected from viral, yeast or human cell lines.

7. The method according to claim 1, wherein the expression vector is a baculovirus system.

8. The method according to claim 1, wherein the steroid receptor is selected from the group consisting of progesterone receptor, glucocorticoid receptor, and mineralocorticoid receptor.

9. The method according to claim 1, wherein the steroid receptor is glucocorticoid receptor.

10. The method according to claim 1, wherein the heat shock protein is selected from the group consisting of hsp90, hsp70, hsp60 and hsp23.

11. The fluorescently-labeled probe according to claim 1, wherein the emission of said fluorescent label is at least about 450 nm to 700 nm.

12. The fluorescently-labeled probe according to claim 1, wherein the emission of said fluorescent label is at least about 550 nm to 700 nm.

13. The method according to claim 1 wherein the range of fluorescence polarization values in step (a) is obtained by periodically adding increasing amounts of expression vector lysate.

14. The method according to claim 13 wherein the increasing amounts of expression vector lysate is added until no further significant change in polarization value is observed.

15. A fluorescence polarization assay for determining whether a compound binds steroid receptor comprising a library of test compounds comprises the following steps:
(a) determining the fluorescence polarization values of a free fluorescently-labeled probe and the fluorescently-labeled probe bound to an expression vector lysate wherein the lysate comprises a steroid receptor associated with at least three heat shock proteins (hsps) to obtain a range of fluorescence polarization values and selecting a reference fluorescence polarization value falling within that range;
(b) mixing the fluorescently-labeled probe with the lysate in step (a) in a buffered aqueous solution;
(c) adding test compounds to a plurality of containers;
(d) adding the mixture obtained in step (b) to said plurality of containers, and incubating the resulting mixtures of fluorescently-labeled probe, lysate, and test compounds;
(e) measuring the fluorescence polarization values of the incubated mixtures obtained in step (d) to obtain test fluorescence polarization values; and
(f) determining the differences between the test fluorescence polarization values and the reference fluorescence polarization value;
wherein the differences in fluorescence polarization values obtained in step (f) indicate whether the test compounds bind steroid receptor.

16. The method according to claim 15, wherein the probe is dexamethasone or mifepristone.

17. The method according to claim 15, wherein the fluorescent label is rhodamine or a rhodamine derivative.

18. The method according to claim 15, wherein the fluorescently-labeled probe is tetramethylrhodamine-dexamethasone.

19. The method according to claim 15, wherein the fluorescently-labeled probe is tetramethylrhodamine-mifepristone.

20. The method according to claim 15, wherein the expression vector is selected from viral, yeast or human cell lines.

21. The method according to claim 15, wherein the expression vector is a baculovirus system.

22. The method according to claim 15, wherein the steroid receptor is selected from the group consisting of progesterone receptor, glucocorticoid receptor, and mineralocorticoid receptor.

23. The method according to claim 15, wherein the steroid receptor is glucocorticoid receptor.

24. The method according to claim 15, wherein the heat shock protein is selected from the group consisting of hsp90, hsp70, hsp60 and hsp23.

25. The fluorescently-labeled probe according to claim 15, wherein the emission of said fluorescent label is at least about 450 nm to 700 nm.

26. The fluorescently-labeled probe according to claim 15, wherein the emission of said fluorescent label is at least about 550 nm to 700 nm.

27. The method according to claim 15, wherein the range of fluorescence polarization values in step (a) is obtained by periodically adding increasing amounts of expression vector lysate.

28. The method according to claim 27 wherein the increasing amounts of expression vector lysate is added until no further significant change in polarization value is observed.

29. A fluorescence polarization assay for determining whether a compound binds steroid receptor comprising:
(a) determining the fluorescence polarization values of a free fluorescently-labeled probe and the fluorescently-labeled probe bound to an expression vector lysate, wherein the lysate comprises a steroid receptor associated with at least three heat shock proteins (hsps) to obtain a range of fluorescence polarization values and selecting a reference fluorescence polarization value falling within that range;
(b) mixing the fluorescently-labeled probe with the lysate in step (a) in a buffered aqueous solution;
(c) mixing a test compound dissolved in a buffered aqueous solution with the mixture obtained in step (b) and incubating the resulting mixture of fluorescently-labeled probe, lysate, and test compound;
(d) measuring the fluorescence polarization value of the incubated mixture obtained in step (c) to obtain a test fluorescence polarization value; and
(e) determining the difference between the test fluorescence polarization value and the reference fluorescence polarization value;
wherein the difference in fluorescence polarization values obtained in step (e) indicates whether the test compound binds the steroid receptor.

30. The method according to claim 29, wherein the probe is dexamethasone or mifepristone.

31. The method according to claim 29, wherein the fluorescent label is rhodamine or a rhodamine derivative.

32. The method according to claim 29, wherein the fluorescently-labeled probe is tetramethylrhodamine-dexamethasone.

33. The method according to claim 29, wherein the fluorescently-labeled probe is tetramethylrhodamine-mifepristone.

34. The method according to claim 29, wherein the expression vector is selected from viral, yeast or human cell lines.

35. The method according to claim 29, wherein the expression vector is a baculovirus system.

36. The method according to claim 29, wherein the steroid receptor is selected from the group consisting of progesterone receptor, glucocorticoid receptor, and mineralocorticoid receptor.

37. The method according to claim 29, wherein the steroid receptor is glucocorticoid receptor.

38. The method according to claim 29, wherein the heat shock protein is selected from the group consisting of hsp90, hsp70, hsp60 and hsp23.

39. The fluorescently-labeled probe according to claim 29, wherein the emission of said fluorescent label is at least about 450 nm to 700 nm.

40. The fluorescently-labeled probe according to claim 29, wherein the emission of said fluorescent label is at least about 550 nm to 700 nm.

41. The method according to claim 29, wherein the range of fluorescence polarization values in step (a) is obtained by periodically adding increasing amounts of expression vector lysate.

42. The method according to claim 41, wherein the increasing amounts of expression vector lysate is added until no further significant change in polarization value is observed.
